(19) United States
(12) Patent Application Publication
(10) Pub. No.: US 2017/0095504 A1
(43) Pub. Date: Apr. 6, 2017

(54) Inhibitors of Kidney Stone Formation and Calcium Deposition

(71) Applicant: Shantha Sarangapani, Norwood, MA (US)

(72) Inventor: Shantha Sarangapani, Norwood, MA (US)

(21) Appl. No.: 15/287,490

(22) Filed: Oct. 6, 2016

Related U.S. Application Data
(60) Provisional application No. 62/237,991, filed on Oct. 6, 2015.

(51) Int. Cl. A61K 31/795; A61L 31/10; A61L 29/16; A61L 29/08; A61L 27/54; C08G 69/48; A61K 31/198; A61K 31/194; A61K 31/765; A61K 31/785; C08F 8/32; C08G 65/48; A61L 31/16; A61L 27/34

(52) U.S. Cl. CPC A61K 31/795; A61L 31/16; A61L 31/10; A61L 29/16; A61L 29/085; A61L 27/54; A61L 27/34; A61K 47/481; A61K 31/198; A61K 31/194; A61K 31/765; A61K 31/785; C08F 8/32; C08G 65/48; C08G 69/48; A61L 2300/404; A61L 2420/06

(57) ABSTRACT

A pharmaceutical composition for the treatment of pathological calculus and plaque formation is provided. The composition includes conjugates of polymers and metal binding agents that are particularly effective in inhibiting crystal nucleation, growth, and aggregation in the body, such as that observed in the formation of kidney stones. The composition is effective at low dosage, thereby avoiding the side effects typically associated with current treatments for kidney stones. The composition is also effective against plaque formation, and may have intrinsic antimicrobial activity. Also provided are methods of treating diseases and surfaces of devices and materials treated with the composition.

### CROSS REFERENCE TO RELATED APPLICATIONS

This application claims the priority of U.S. Provisional Application No. U.S. 62/237,991 filed on 6 Oct. 2015 and entitled “Antimicrobial Synergistic Potentiators, Calcium Oxalate inhibitors and Formulations”, which is hereby incorporated by reference in its entirety.

### BACKGROUND

Calculus and plaque formation are a significant worldwide issue. The lifetime prevalence of urolithiasis, for example, is 13% for men and 7% for women in the U.S (NIDDK) and an estimated $2.1 billion was spent in claims related to a urolithiasis diagnosis in 2000 (Pearle, 2005). Stones injure kidneys, cause infection and obstruction and may cause intense pain, urinary infection and bleeding. Moreover, up to 50% of patients may have recurrence within 5 years (Asplin et al., 1996), with chances being higher when there is incomplete removal of fragments (Chow et al.; 2004). The recurrence of stones after treatment is normally controlled by diet and/or high doses of oral medications such as thiazides, which facilitates calcium reabsorption by blocking the Na+/Cl− transporter, and potassium citrate, which in large doses reduce calcium ion supersaturation in the urine. Current dosages for these medications are very high and can cause serious side effect such as potentially fatal asymptomatic hyperkalemia and cardiac arrest. Although concern about hypokalemic arrhythmias from thiazide use continues, its preeminence has been replaced by concerns about other metabolic side effects (Ellison et al, 2009). The compliance with alkaline citrate was shown to be less than 50% due to its unpalatability and severe gastrointestinal upset episodes. Therefore, current treatments are based on the alteration of urine biochemistry, by reducing supersaturation to lower the risk of precipitation. Despite the medical and economic importance of urolithiasis, novel therapy agents have not been developed in a long time. The ideal pharmacological agent should thus halt the formation of calcium stones, cause less side effects and be effective at low doses in order to achieve a reasonably good compliance even for a life-long therapy.

Plaque is another form of organic and mineral buildup that may lead to pathological conditions. It has been generally recognized that atherosclerosis is associated with a deposition of calcium in the arterial wall, although the pathogenic significance of this calcification remains unclear. In recent years, a number of calcium-chelating agents and organic calcium antagonists have been shown to exert antiatherogenic effects in animals with dietary hypercholesterolemia. Moreover, plaque or encrustation are a problem affecting biomedical devices as well, such as prosthetics and catheters. These plaques are difficult to prevent or eliminate, and may be further complicated by the growth of bacteria. Bacterial growth in plaques and calculi is resistant to antibacterial agents and is a growing concern. There is pressing need for more effective ways of delivering antimicrobial agents to bacteria growing in these conditions.

There is a need for potent inhibitors for calculus management and infection control.

### SUMMARY OF THE INVENTION

The instant invention provides effective control of calculus and plaque formation. It provides compositions and methods to inhibit nucleation and growth of mineral crystals even when administered in very low doses. The invention includes the use of metal binding agents conjugated to polymers which may also exhibit chelating properties for a synergistic metal sequestering effect. This invention is useful for any medical condition involving calculus or plaque formation, such as kidney stones, including calcium oxalate and infection stones, gall stones, atherosclerosis, and device encrustations where insoluble calcium compound deposits could be harmful. The invention may also exhibit antimicrobial activity and urease inhibition in vivo as well as ex vivo, thus preventing the formation and growth of biofilms and plaques on body tissues and medical devices, tubings, prosthetics, stents, and catheters.

One aspect of the invention is a pharmaceutical composition for the treatment of any disease characterized by calculus and/or plaque formation. The composition includes a conjugate of a polymer with a metal binding or chelating agent.

Another aspect of the invention is a method of treating a disease, the method including administering the above composition to a subject in need thereof.

Yet another aspect of the invention is a method of preventing plaque formation on a surface that includes contacting the composition with the surface.

The invention also can be summarized with the following list of embodiments.

1. A pharmaceutical composition for the treatment of a disease characterized by calculus and/or plaque formation comprising a conjugate comprising a polymer and a metal binding agent.
2. The composition of embodiment 1, wherein the polymer and the metal binding agent are covalently bound.
3. The composition of embodiment 1, wherein the polymer contains metal-binding groups.
4. The composition of embodiment 1, wherein the polymer contains metal-binding groups from 1-60%.
5. The composition of embodiment 1, wherein the metal binding groups are chelating agents.
6. The composition of embodiment 1, wherein the polymer and/or the chelating agent have metal binding groups selected from the group consisting of aminocarboxylates, polyaminocarboxylates, multi-carboxylic acids, hydroxylcarboxylates, polyhydroxyl alcohols, polysaccharides, sulfonates, dithiocarbamates, dialkyl thioesters and derivatives, amino acids, aminosulfonates, phosphonates, macrocyclic ligands with amino or tertiary nitrogen and thiols.
7. The composition of embodiment 1, wherein the metal binding agent binds preferentially to calcium ions.
8. The composition of embodiment 1, wherein the metal binding agent binds preferentially to calcium sites along with polymer adsorption.
9. The composition of embodiment 1, wherein the metal binding agent binds preferentially to calcium sites along with the adsorption of the polymer.
10. The composition of embodiment 1, wherein the composition has antimicrobial activity.
11. The composition of embodiment 1, wherein the metal binding agent preferentially binds to calcium ions in a calcium rich environment.
12. The composition of embodiment 1, wherein the metal binding agent binds to the calcium sites of the calculus or plaque.
13. The composition of embodiment 1, wherein the polymer adsorbs on the nucleating sites.
14. The composition of embodiment 1, wherein the polymer is selected from the group consisting of hyperbranched polyglycerol, multi-arm branched polyethylene glycols, polyvinyl sulfonates, polyvinylamine, polyvinyl alcohols, N-(2-hydroxypropyl) methacrylamide, polyamino acids, and combinations thereof.
15. The composition of embodiment 1, wherein the polymer is selected from the group consisting of hyperbranched polymers such as polyglycerol, multi-arm, branched polyethylene glycols, poly vinylsulfonates, polyvinyamine, polyvinylalcohols, N-(2-hydroxypropyl) methacrylamide, polyamino acids such as poly-L-lysine, and combinations thereof.
16. The composition of embodiment 1, wherein the polymer is polyvinyl sulfonate.
17. The composition of embodiment 1, wherein the polymer is a polyamino acid with peptide linkages.
18. The composition in embodiment 1 where in the polymer is a nanosize hyperbranched polymer with a size of less than 100 nm and containing hydroxyl, alcohol, amino or carboxylate groups.
19. The composition of embodiment 1, wherein the chelating agent is selected from the group consisting of diethylenetriaminepentaacetic acid, citrate, sulfonates and combinations thereof.
20. The composition of embodiment 1, wherein the chelating agent is diethylenetriaminepentaacetic acid.
21. The composition of embodiment 1, wherein the polymer is polyvinyl sulfonate and the chelating agent is diethylenetriaminepentaacetic acid.
22. The composition of embodiment 1, wherein the composition is suitable for oral, topical or parenteral administration, controlled delivery, three dimensional printed forms, transdermal patches, and microneedle delivery.
23. A method of treating a disease, the method comprising administering the composition of embodiment 1 to a subject in need thereof.
24. The method of embodiment 23, wherein the composition acts by inhibiting crystal formation and/or growth by adsorption.
25. The method of embodiment 23, wherein the disease is urolithiasis.
26. The method of embodiment 23, wherein the disease is calcium urolithiasis or struvite urolithiasis.
27. The method of embodiment 23, wherein the disease is atherosclerosis.
28. The method of embodiment 23, wherein the disease is microbial plaque or calculus.
29. A method of inhibiting plaque formation on a surface comprising contacting the composition of embodiment 1 with said surface.
30. The method of embodiment 29, wherein the composition inhibits growth or formation of a biofilm.
31. The method of embodiment 19, wherein the surface to be treated is part of medical device.
32. The method of embodiment 29, wherein the medical device is a prosthetics, stent or catheter.

### BRIEF DESCRIPTION OF THE DRAWINGS

FIG. 1 shows induction times data for various inhibitors in comparison to citrate.

FIG. 2 shows induction times data for PVS and citrate.

FIG. 3 shows inhibition efficiency of various inhibitors per concentration in mass units.

FIG. 4 shows inhibition efficiency of various inhibitors per molar concentration.

FIGS. 5A-B show the inhibition of crystal growth and aggregation in human urine in the presence of inhibitors PVS-DTPA (coded as KSI-02A, FIG. 5A) and HPG-DTPA (coded as KSI-02, FIG. 5B).

### DETAILED DESCRIPTION OF THE INVENTION

The present invention provides composition and methods for inhibiting nucleation and growth of mineral crystals in the body or medical devices. The invention acts by directly inhibiting crystal formation at low doses via multisite adsorptive inhibition. The rates of inhibition (kinetic) as well as the effect on the equilibrium aspects play key roles depending on the application. A kinetic inhibitor does not necessarily reduce crystal yields. The compositions delay the nucleation rate and reduce the yield of crystals, as well as the aggregation of crystals to form larger particles. Certain compositions also reduce the amount of crystals formed in addition to kinetic inhibition. The present compositions may have the ability at the molecular level to penetrate the barriers and achieve glomerular concentrations. The compositions may also reduce protein adsorption on crystals, thus further inhibiting crystal growth. In the instant invention, cooperativity of both the polymer chain and the conjugated pendant groups synergistically contribute to the adsorptive inhibition effects and calcium binding on crystal sites when compared to the polymer alone or the functional group moiety alone.

In one embodiment, the invention is a pharmaceutical composition for the treatment of a disease characterized by calculus and/or plaque formation including a conjugate including a polymer and a metal chelating agent. In preferred embodiments, the disease is urolithiasis. In this application, urolithiasis and nephrolithiasis are used interchangeably to indicate any calculus formation along the urinary tract, including calculi in the kidneys, within the ureter, passed into the bladder, and obstructing the ureteropelvic junction. In a preferred embodiment, the composition prevents the nucleation and growth of calcium oxalate (CaOx) crystals, which causes over 70% of kidney stones. In preferred embodiments, the composition shows activity in inhibiting the growth and aggregation of CaOx crystals in human urine.

Calculus can be any hard mineral deposit formed in an organ or tissue. Calculi can be formed by minerals and/or acid salts. Calculi can be any concretion of material that forms in an organ or duct of the body causing a medical condition, such as kidney stones and gallstones. Plaque or encrustation can be formed on a surface by accumulation of various substances, such as fat, mineral crystals, proteins and microbial films. By inhibiting the nucleation rate and aggregation rate, the basic foundation of the calculus or plaque formation is drastically reduced or prevented.

In some embodiments, the polymer and metal chelating agent are covalently bound. In some embodiments, they are bound by electrostatic interactions. In preferred embodiments, the polymer contains metal-binding groups, thereby acting as a metal chelator. Chelating agents are used to sequester metal ions thus avoiding or minimizing undesired effects of free metal ions. In some embodiments, the polymer and/or the chelating agent have metal binding groups selected from the group consisting of polyamino carboxylates, polyamino acids, multi-carboxylic acids, hydroxyl poly carboxylates, sulfonates, dithiocarbamates, oxo-dithiocarbamates and derivatives, amino acids, macrocyclic ligands with amino or tertiary nitrogen and thiols. In preferred embodiments, the polymer and/or the chelating agent have multiple carboxylic and/or sulfonate groups that can act as metal chelators. In some embodiments, the metal chelating agent binds preferentially to calcium ions. Different polymers and chelators may act by different mechanisms. Some may not bind calcium but adsorptively delay the calcium oxalate formation without reducing the crystal yield, whereas others may reduce the crystal yield drastically. Combination of molecules acting by different mechanisms may be very effective achieving both long induction times and high inhibition efficacy in human urine. The conjugation of PVS with DTPA or crosslinked/copolymerized with a cationic HPMA can be very effective achieving both long induction times and high inhibition efficacy in human urine. In preferred embodiments, there is a synergistic effect between the chelating agents and the metal-binding polymers.

In some embodiments, the polymer and/or the composition exhibit extremely high selectivity for the kidney. In some embodiments, the composition has antimicrobial activity. In some embodiments, the composition includes antimicrobial and/or urease agents, such as silver and DTPA, thus exhibiting further benefits for controlling bacterial and protein biofilms that promote nucleation of mineral crystals.

Even though organic content is kidney stones is only 2-3%, some proteins present in urine facilitate the aggregation of kidney calcium oxalate stone crystals. Therefore, it is important for the inhibitor polymeric species to reduce protein adsorption. The surfactant-like features of the instant invention with hydrophilic organic and ionic moieties such as the SO³⁻ could significantly reduce protein accumulation on the existing stones. Generally, polyethylene oxide, HPG and PEG based surfaces are well-known to resist protein adsorption. Hyperbranched polyglycerols also resist the nonspecific adsorption of proteins. Hyperbranched polymers, when conjugated to a therapeutic agent, provide protection to the therapeutic agent and could provide a readily absorbable form of the therapeutic agent in the intestines. In fact, compared to biological macromolecules simple polymer structures such as the instant invention could have better protective barrier type adsorption. In contrast to biological macromolecules, a high number of chelating groups can interact strongly with the respective binding region, which results in a significant increase of binding affinity for the polymer. In this respect the synthetic macromolecules could effectively compete with proteins and have a strong advantage due to the favorable thermodynamic factors due to the multisite adsorption forces. Without being bound by any theory, polymeric compounds may delay crystal formation significantly and also passivate any crystal surfaces to prevent them from adhering to cells or other crystals (aggregation). In some embodiments, the composition has long-term adsorption activity and can be administered daily, weekly or less frequently. In some embodiments, the composition prevents protein adsorption to calculi and plaque, further inhibiting crystal growth.

In some embodiments, the polymer is selected from the group consisting of hyperbranched polyglycerol (HPG), multiarm polyethylene glycols, amino bearing polymers, poly vinyl sulfonates, poly N-(2-hydroxypropyl) methacrylamide (HPMA), poly-L-lysine (PLL), and combinations thereof. In preferred embodiments, the polymer is a nanopolymer, polyvinyl sulfonate (PVS), poly-L-lysine, and combinations thereof. In some embodiments, the chelating agent is selected from the group consisting of diethylene triamine pentacetate (DTPA), citrate, sulfonates and combinations thereof. In preferred embodiments, the chelating agent is DTPA. In certain embodiments, the polymer is polyvinyl sulfonate and the chelating agent is DTPA. DTPA conjugation chemistry to hydroxyl and amino functionalities is well understood with significant evidence of longer half-lives and biocompatibility with highly researched macromolecular-chelate contrast agents provide confidence on the bioavailability and stability of our conjugates with half-lives suitable for entering the circulation and renal clearance.

In some embodiments, the composition is a pharmaceutical formulation suitable for oral, topical or parenteral administration.

In some embodiments, the polymer is a nanosize (i.e., less than 100 nm) hyper branched polyglycerol conjugated to DTPA or to citrate. In some embodiments, the polymer is polyvinylsulfonate conjugated to DTPA and copolymerized/crosslinked with low molecular weight N-(2-hydroxypropyl) methacrylamide, nanosized. In some embodiments, the polymer is poly-L-lysine conjugated with DTPA, citrate and or sulfonates. In some embodiments, the polymer is a vinylsulfonate copolymerized with acrylic acid and/or vinyl amines. In some embodiments, the polymer includes alginates, chitosan and/or polyethylene imines derivatizations conjugated with citrate, DTPA and/or sulfonates. In some embodiments, the polymer includes carboxy polysaccharides derivatizations conjugated with DTPA and/or citrate. In some embodiments, DTPA or citrates are covalently bonded with a polymerizable moiety.

In some embodiments, the polymers can have a minimum molecular weight of 600 Daltons. In other embodiments, the polymers can have a maximum molecular weight of 100 kDa. In some embodiments, nanosized and/or flexible polymeric entities can overcome the intestinal absorption barrier despite their high molecular weight and enter the blood stream. The threshold for renal filtration of polymers corresponds very roughly with molecular weights ranging from 30 kDa to 50 kDa depending on the polymer chemistry, shape, molecular conformation, and flexibility. Thus, the MW of a polymer plays a critical role in polymer pharmacokinetics and biodistribution. However, this limitation could be circumvented by a careful choice of molecular weight, flexibility, molecular conformation. Nano structures such as the HPGs and HPMA as carriers offer 2-5 nm size an attractive potential target for overcoming the glomerular barrier.

In some embodiments, the present invention provides methods for treating a disease using the above-mentioned composition. In some embodiments, the composition acts by inhibiting crystal formation and/or crystal growth by adsorption.

In some embodiments, the disease is urolithiasis. Adhesion of newly formed calcium oxalate monohydrate (COM) crystals to the apical surface of renal tubular epithelial cells could be an important initiating event of stone formation and may be reduced or prevented by the present invention. In some embodiments, the disease is calcium urolithiasis or struvite urolithiasis. In some embodiments, the composition is effective against struvite by a combination of chelating and antimicrobial properties.

In some embodiments, the composition is effective against plaque formation in the body. Plaque may be formed within blood vessels or in and/or around oral structures. In some embodiments, the disease is atherosclerosis. In some embodiments, the disease is dental plaque and/or dental calculi.

In some embodiments, the present invention provides methods for inhibiting plaque formation on a surface using the above-mentioned composition. In some embodiments, the composition is effective against ex vivo plaque formation. In some embodiments, the composition inhibits growth or formation of a biofilm, such as bacterial biofilms. In some embodiments, the surface to be treated is part of medical device, such as prosthetics and catheters. In some embodiments, the composition is formulated as a coating into and/or onto medical devices.

### Examples

### Example 1. Material and Methods

### Synthesis of PVS Polymer

PVS polymer was synthesized from vinyl sulfonate using a 1% radical initiator such as persulfate at about 50-60° C. in distilled water in nitrogen atmosphere. The product is precipitated in acetone and purified by dialysis with a suitable cutoff. The average molecular weight was about 2000-2500. This product is also commercially available.

Synthesis of Hyperbranched Polyglycerol (HPG) Conjugated to Citric Acid or DTPA.

A solution of diethylenetriaminepentaacetic acid in a dry solvent was stirred at 65° C. for 1 hour. The reaction mixture was cooled to room temperature (RT) and then mixed with an appropriate amount of HPEG, plus N,N-diisopropylethylamine in DMF and allowed to react. HPG was dissolved in dioxane at a final concentration of 10 mg/ml and dropped into 5 ml of dimethylsulfoxide (DMSO) containing 143 mg of DTPA anhydride and 24 mg of 4-(dimethylamino)pyridine (DMAP). The reaction solution was magnetically stirred at 40° C. for 6 h, followed by precipitation by diethyl ether. The precipitate was collected by centrifugation (5000 rpm at 20° C. for 10 min), and dissolved in dioxane. This process was repeated 3 times to obtain DTPA-introduced HPEG.

Another method to perform the reaction was conducted as follows. To a mixture of BPEG (420 mg, 0.1 mmol, 1 eq) and DTPA anhydride (714 mg, 2 mmol, 20 eq) in DMF (15 mL) was added DMAP (20 mg, 0.2 mmol). The mixture was heated at 140° C. for 3 days and concentrated to give semi-solid/emulsion. The stuff was taken up into water (15 mL) (pH=˜3) to heat at 100° C. for 2 days. A pale yellow solution was obtained. HPG (1 mol) dissolved in water reacted with H5IO6 (100 mol) added dropwise under stirring. After 1 h the solution was dialyzed against water (2.5 L, 1000 MWCO) over night and the water changed once. 35-50 mol of DTPA amide was added to the reaction mixture and the solution stirred for 1 h, after which ethanolamine (10% in water) was added and solution stirred for another 1 h. NaBH3CN (238 mol) was dissolved in 200 L water, added to the mixture and stirred for >6 h. The reaction mixture was dialyzed for 5 days against water (2.5 L). The product is a syrupy liquid and was precipitated by solvents such as THF. The precipitate was collected by centrifugation and washed with dioxane/water.

Sulfonate modifications of hyperbranched polygycerols (HPG).

HPG was synthesized using three different methods. In the first method, star-shaped poly(ethoxyethyl glycidyl ether) and triethylamine (3.2 mL, 24 mmol) were dissolved in dichloromethane (20 mL) and cooled to 0° C. Afterwards, 2-chloroethylsulfonyl chloride was added and the solution warmed up to room temperature (RT) while stirring for 60 min. The reaction is terminated by addition of sodium carbonate solution. The organic phase was washed twice with sodium carbonate solution and twice with water and then dried over magnesium sulfate. After removal of the solvent a highly viscous brownish liquid was obtained. In the second method, a known amount of the hyperbranched polyglycerol was dissolved in water. The reaction solution was cooled to approximately 5° C. and a stoichiometric amount of sodium salt of vinylsulfonic acid in form of a 25% by weight, aqueous solution is slowly added for 4 hours via a dropping funnel and polymerized using potassium persulfate as the initiator. After the addition was completed the reaction mixture was brought to RT and stirred for another 3 days. After removing the solvent in vacuum, the obtained raw product was further purified by dialyzing in water for 24 hours, wherein the water was changed three times. The raw product is concentrated in vacuum and dried for removing the remaining water in a desiccator. In the third method, vinyl sulfonate was polymerized by anionic initiators on polyhydroxy natural compounds such as cellulose, starch, cotton, lignin and their derivatives, and on synthetic polymers by using metal alkoxide. After polymerization was carried out, the product obtained was characterized by LC-MS and Maldi ToF.

Attachment of DTPA to Hyperbranched Polyglycerol.

HPG (1 mol) dissolved in water reacted with H5IO6 (100 mol) drop wise under stirring. After 1 h the solution was dialyzed against water (2.5 L, 1000 MWCO) over night and water was changed once. 35-50 mol of DTPA amide was added to the reaction mixture and the solution was stirred for 1 h followed by ethanolamine (10% in water) and solution stirred for another 1 h. NaBH3CN (238 mol) was dissolved in 200 L water, added to the mixture and stirred for at least 6 h. The reaction mixture was dialyzed for 5 days against water (2.5 L). The product is a syrupy liquid and was precipitated by solvents such as THF. The precipitate was collected by centrifugation and washed with dioxane/water.

Synthesis of Polylysine-DTPA.

The cyclic anhydride of DTPA (Sigma-Aldrich) in DMF is reacted with PLL (250 lysine units) in sodium bicarbonate at pH=9 at a ratio of 6 mmoles of lysine to 2.5 mmoles of anhydride and stirred for 2 hrs at 0° C. and at room temperature. The product was concentrated under vacuum and dialyzed against PBS buffer at 4° C. with several changes of buffer. The product was precipitated by adding ethanol and checked by TLC, NMR. In another method, polylysine is reacted with DTPA difluorophenyl ester tetra-t-butyl ester (Macrocyclics, TX) at RT for several hours. The reaction solution was evaporated and the product was precipitated using diethyl ether. Purification by dialysis gives a colorless solid

Synthesis of PLL-DTPA.

The cyclic anhydride of DTPA in DMF was reacted with PLL (250 lysine units) in sodium bicarbonate, pH=9, at a ratio of 6 mmol of lysine to 2.5 mmol of anhydride and stirred for 2 hours at 0° C. and at room temperature. The product was concentrated under vacuum and dialyzed against PBS buffer at 4° C. with several changes of buffer. The product is precipitated by adding ethanol and checked by TLC. This syntheses reaction resulted in DTPA attachment of the order of 4-5/mol of HPG as verified by the neutralization of the highly acidic product, calcium uptake and approximate mol-weight determination by MALDI-TOF. The HPG alone has 68 hydroxyl groups (MALDI TOF with an approximate peak at 5000 and the DTPA/HPG is only 5-6 moles/mole of HPG. Higher molecular weight HPGs should provide more DTPA surface groups.

Synthesis of Polyvinylsulfonate PVS-DTPA Copolymerized/Crosslinked with an Inert Biocompatible Cationic Copolymer N-(2-Hydroxypropyl)Methacrylamide.

PVS-DTPA conjugate lightly crosslinked with a cationic copolymer N-(2-hydroxypropyl)methacrylamide (HPMA)-nanosized (5-10 nm) water-soluble constructs (solid dispersed in the submicron range) is shown below:

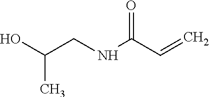

HPMA is shown below:

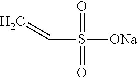

It can be copolymerized to bridge the PVS with DTPA in solution via normal radical polymerization conditions at 65° C. for 20 hours using AlBN as a thermal initiator to form a series of copolymers by reversible addition-fragmentation chain transfer (RAFT) reaction, which allow hyperbranched poly HPMA as well. At low concentrations the polymers are anticipated to be in the random coil form surrounding the DTPA. The purification of the compounds are by dialysis, mass spectrometry and light scattering for particle size distribution in physiological media

### Example 2. Induction Times for Polymers, Chelators and Conjugates

The period of time elapsing between the achievement of super saturation and the observation of the appearance of a new solid phase (i.e. nuclei) or crystals, known as the induction or the initiation period, is often a sensitive measure of the effectiveness of an additive in inhibiting crystallization. The experiments were performed in aqueous medium with equimolar concentrations of calcium and oxalate, each close to 0.5 mM. High molecular weight (HMw) PLL (30 kDa-40 kD) had DTPA content of ˜50% w/w. Low molecular weight (LMw) PLL (1-5 kDa) also had DTPA content of ˜50% w/w. HPG-DTPA had DTPA content of about ˜30% w/w. The total reaction mass was 100 gm. An aqueous solution of calcium chloride dihydrate with inhibitor (at desired concentration) was prepared amounting to 99 gm. The conductivity probe was inserted into the solution and conductivity value was noted. To this solution, 1 gm of 0.05 M sodium oxalate solution was added at time t=0 sec. The conductivity values were noted every 30 sec. During the experiment, the conductivity values remained stable (after an initial increase because of oxalate addition) for a short duration and started to decrease as the nucleation proceeded. This period of time was recorded as induction time. The induction time values are +/−30 sec (since the conductivity values are recorded every 30 sec). The conductivity values for the experiments performed were in the range of 0-300 μS/cm. FIG. 1 indicates the induction times for each inhibitor concentration.

### Example 3. Induction Times for PVS Vs. Citrate

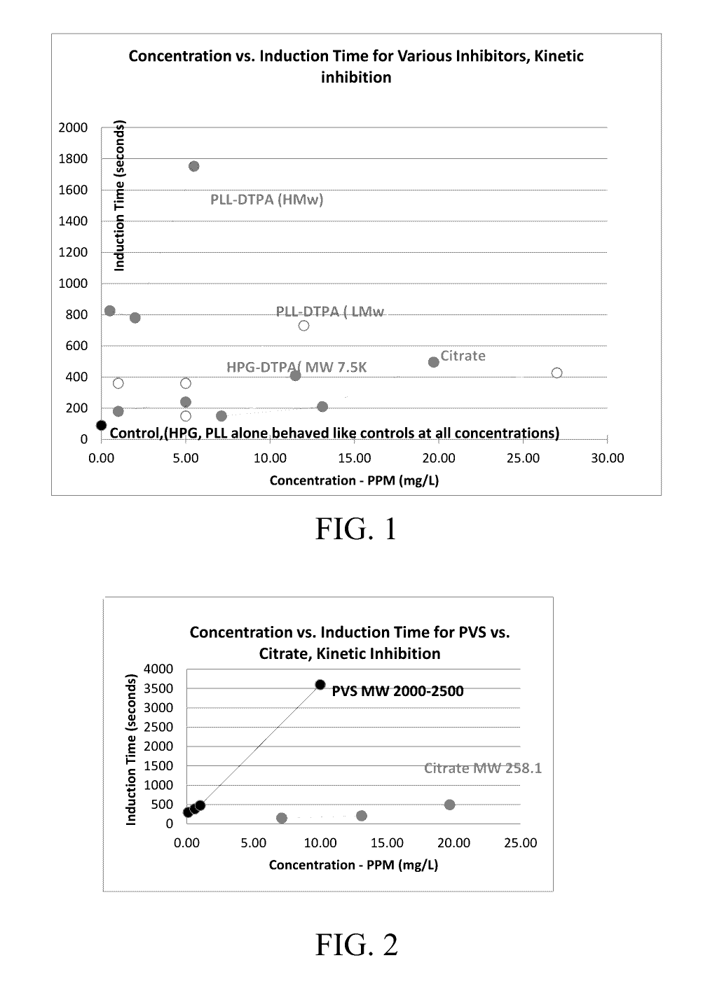

The experiments were performed in aqueous medium with equimolar concentrations of calcium and oxalate, each close to 0.5 mM. A calibrated conductivity probe was inserted into the solution and conductivity value was noted. We report a synergistic effect that was reproducibly observed when conjugation of anionic DTPA with hyper branched polyglycerols (HPG-DTPA), poly-lysine polymers (PLL-DTPA) or polysulfonates (PVS-DTPA) provided a superior effect compared to the DTPA or citrate alone. The DTPA mass percentages in the various polymer bound mode were 30-50% compared to 100% DTPA alone. Polysulfonate provided longer induction times (delaying the formation of calcium oxalate nucleation) at low concentrations (less than 100 PPM) (FIG. 2).

### Example 4. Determination of Inhibition Efficiencies Per Concentration in Mass Units

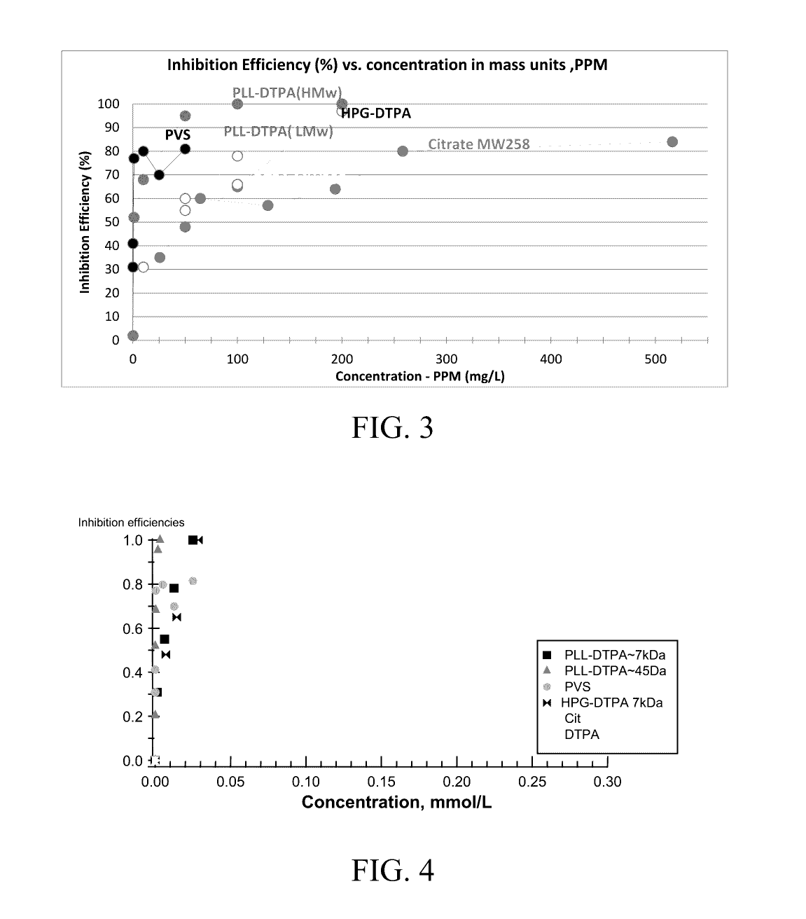

The desupersaturation curves ([Ca2+] free with respect to time) during calcium oxalate precipitation were fit to a curve using cubic smoothing spline function (‘csaps’) in MATLAB (FIG. 3). The precipitation rate (d [Ca2+] free/dt) was obtained from the first derivative of the curve. The experiments were performed with an overall calcium and oxalate concentrations close to 1 mM. The additive concentrations were varied. Experiment was conducted using a buffer containing 10 mM HEPES and 150 mM NaCl with a pH of ˜7.3-7.6 (adjusted using 0.1 M NaOH) The concentration of free Ca2+ during the crystallization experiment was monitored by a Ca2+ selective electrode and connected to a computer. The precipitation rate (d [Ca2+] free/dt) was obtained from the first derivative of the curve. Maximum growth rates with and without additive obtained from the first derivative curve. Inhibition efficiency (T) is calculated from the maximum growth rate obtained as follows: I=(R₀−R)/R₀ where ‘R’ and ‘R₀’ are the maximum growth rates with and without additive respectively.

### Example 5. Determination of Inhibition Efficiencies in Molar Concentration

The desupersaturation curves were obtained as in the previous example. FIG. 4 shows the mg/L converted to molar concentrations. Molarities were calculated on the basis of the molecular weight determinations by MALDI-TOF and/or correlations with titrations of the DTPA groups in the polymer with calcium using a calcium electrode. The mmoles of calcium binding per unit weight of the polymer DTPA was then calculated. Clearly, the polymeric conjugates showed inhibition efficacy at 10-40 micromolar levels.

### Example 6. Effect of Additives on Seeded Calcium Oxalate Crystal Growth and Effect of Proteins in Human Urine

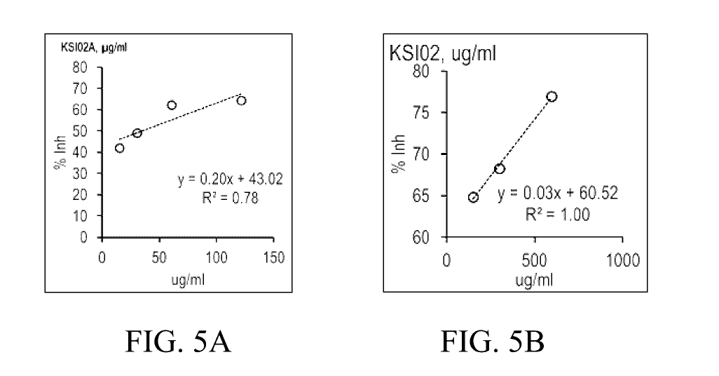

Inhibitors were evaluated in concentrated human urine samples with a protein (albumin) content of about 30-40%. Calcium oxalate crystal growth was measured in quadruplicate using a 96 well microplate seeded crystal growth assay measuring crystal growth with light scattering (wavelength=450 nm), where light scattering is shown to have direct correlation to crystal mass. The assay conditions were 10 mM Ca, 1 mM Ox, 25 mM Tris, 150 mM NaCl, pH 7.2, and 120 μg/ml COM seed crystals. These assay conditions mimicked the average urinary calcium to oxalate ratios (10:1) and seed crystals additions were at a concentration that favored crystal growth over nucleation (seed crystal additions <50 μg/ml) or growth-related crystal aggregation (seed crystal additions >300 μg/ml) (FIGS. 5A-B). Results are summarized in Table 1.

TABLE 1
Effect of PVS-DTPA and HPG-DTPA on
Seeded Calcium Oxalate Crystal Growth.

| | | |
|---|---|---|
| PVS- | p < 0.001 | Significant inhibition at all concentrations: |
| DTPA | | 15, 30, 61, 122 μg/ml → 42, 49, 62, 64% Inhibition |
| HPG- | p = 0.001 | Significant inhibition; test at intermediate |
| DTPA | | concentrations: 149, 298, 596 μg/ml → 65, 68, |
| | | 77% Inhibition |

### Example 7. Effect of Inhibitors on Crystal Morphology

The crystals used for characterization are samples obtained 1 hr after the start of the reaction (at the end of kinetic experiments), filtered, and dried for at least 24 hrs. Crystals were classified into calcium oxalate monohydrate (COM), calcium oxalate dihydrate (COD) or calcium oxalate trihydrate (COT). COT is generally reported as an unstable phase which eventually transforms to COD or COM the latter being the most stable phase. Results obtained were: control (no inhibitor) yields COM; PVS 25 mg/L: COD, COT; HPG-DTPA: 50 mg/L, 100 mg/L COT; PLL-DTPA 10 mg/L: COT; PLL-DTPA 100 mg/L: COT; DTPA 100 mg/L: COT.

### Example 8. Calcium Binding Data

Calcium binding data and polymorphic outcomes are provided in Table 2. Calcium binding was determined before oxalate addition. Crystals were obtained after the end of the kinetic experiments (1 hour), filtered, dried and tested by FTIR. The major polymorph was reported; other phases may be present but their peaks were not distinguishable in IR. Initial calcium and oxalate concentrations was the same, approximately 1 mM in pH buffered medium containing 10 mM HEPES and 150 mM NaCl.

TABLE 2
Calcium binding data and polymorph obtained at different
PVS polymer concentrations average Mw 2000-2500.

| PVS | | | |
|---|---|---|---|
| concentration | Free Ca in solution | Free Ca after | Polymorph by |
| (mg/L) | before PVS (mM) | addition of PVS | FTIR |
| 0.0 (control) | — | — | COM |
| 0.0001 | 1.03 | 1.03 | COT |
| 0.01 | 1.03 | 1.04 | COT |
| 1 | 1.04 | 1.03 | COD |
| 10 | 1.04 | 1.02 | COD |
| 25 | 1.04 | 0.94 | COD |
| 50 | 1.03 | 0.87 | COD |

Table 3 shows that some calcium binding occurs with some of the inhibitors. The data are Ca recordings obtained from solutions containing the inhibitor (100 mg/L) and calcium. The Ca ion concentration should be ˜1.0 mM if no Ca binding occurs. This could be related to the type of adsorption on the crystals as reported in a later section. Some of our proposed KSIs are showing significant Ca binding activity, and these are likely to be the best candidates as inhibitors.

TABLE 3
Calcium binding in the presence of inhibitors.

| | Ca2+ concentration in |
|---|---|
| | the presence of inhibitors |
| Sample | at 100 mg/L (PPM) |
| Control | 1.02 mM |
| PVS(MW 2000-2500) | 1.01 mM |
| HPG-DTPA | 0.83 mM |
| Linear PEG-sulfonate | 1.02 mM |
| HPG-sulfonate | 0.96 mM |
| DTPA alone (99% DTPA) | 0.71 mM |
| PLL-DTPA (30-40 kDa mol weight) | 0.19 mM |
| PVS-DTPA | 0.25 mM |

### Example 9. Adsorption Behavior of Inhibitors

Knowing that preferential adsorption of inhibitors on the growing crystal affect the growth, aggregation and morphological phase changes we evaluated the roles of the proposed inhibitors that modulate the interfacial interactions between additives and crystal surfaces. Adsorption models used for fitting the data of inhibition efficiency were classified into the following different adsorption categories: Langmuir kink, Langmuir terrace, Temkin kink, Temkin terrace. The inhibitors from this invention alter the growth rates by adsorbing onto crystal surfaces of calcium oxalate. The data obtained are for the overall growth rate of the crystals and not for a particular face (polymorph can play an important role). From the correlation coefficients for straight line fits for inhibitors HPG-DTPA and PLL-DTPA, it was inferred that these inhibitors follow Langmuir surface terrace adsorption model. Polymer PVS data and correlation coefficients suggest that PVS follows the Temkin model. The difference of ‘Q0diff (initial differential heat) for Temkin kink is significant for inhibitor PVS compared to other inhibitors suggesting that this follows a Temkin kink adsorption model, in agreement with the findings by Kirborga et al., 2010.

### REFERENCES

1. Pearle M S, Calhoun E A, and Curhan G C. Urologic diseases in America project: urolithiasis. J Urol. (2005), 173; 848-57.
2. Pearle M S, Roehrborn C G, Pak C Y. Meta-analysis of randomized trials for medical prevention of calcium oxalate nephrolithiasis. J Endourol (1999), 13: 679-85 Ardestani et al, J Radioanal Nucl Chem (2010) 283:447-455
3. Asplin J R, Favus M J, Coe F L. Nephrolithiasis. In: Brenner B M, ed. Brenner and Rector's the kidney. 5th ed. Philadelphia: Saunders, 1996: 1893-935.
4. Chow K. et al. Citrate inhibits growth of residual fragments in model of CaOx stones. KidneyInternational, (2004), 65: 1724-1730.
5. Chow K, Dixon J, Gilpin S, et al. A stone farm: development of a method for simultaneous production of multiple calcium oxalate stones in vitro. Urol Res (2004), 32: 55-60.
6. Ellison D H, Loffing J. Thiazide Effects and Adverse Effects: Insights From Molecular Genetics. Hypertension. (2009), 54; 196-202.
7. Kirboga S and Oner M. Colloids and Surfaces B. Biointerfaces (2010) 78: 357-362.

What is claimed is:

1. A pharmaceutical composition for the treatment of a disease characterized by calculus and/or plaque formation, the composition comprising a conjugate comprising a polymer and a metal binding agent.

2. The composition of claim 1, wherein the polymer and the metal binding agent are covalently linked.

3. The composition of claim 1, wherein the polymer contains one or more metal-binding groups.

4. The composition of claim 1, wherein the polymer and/or the chelating agent comprise metal binding groups selected from the group consisting of aminocarboxylates, polyaminocarboxylates, multi-carboxylic acids, hydroxylcarboxylates, polyhydroxyl alcohols, polysaccharides, sulfonates, dithiocarbamates, dialkyl thioesters, amino acids, aminosulfonates, phosphonates, macrocyclic ligands with amino or tertiary nitrogen, and thiols.

5. The composition of claim 1, wherein the metal binding agent binds preferentially to calcium ions.

6. The composition of claim 1, wherein the composition has antimicrobial activity.

7. The composition of claim 1, wherein the polymer is selected from the group consisting of hyperbranched polyglycerol, multi-arm branched polyethylene glycols, polyvinyl sulfonates, polyvinylamine, polyvinyl alcohols, N-(2-hydroxypropyl) methacrylamide, polyamino acids, and combinations thereof.

8. The composition of claim 1, wherein the polymer is polyvinyl sulfonate.

9. The composition of claim 1, wherein the chelating agent is selected from the group consisting of diethylenetriaminepentaacetic acid, citrate, sulfonates and combinations thereof.

10. The composition of claim 1, wherein the chelating agent is diethylenetriaminepentaacetic acid.

11. The composition of claim 1, wherein the polymer is polyvinyl sulfonate and the chelating agent is diethylenetriaminepentaacetic acid.

12. The composition of claim 1, wherein the composition is suitable for oral, topical, or parenteral delivery.

13. A method of treating a disease, the method comprising administering the composition of claim 1 to a subject in need thereof.

14. The method of claim 13, wherein the composition acts by inhibiting crystal formation and/or growth by adsorption.

15. The method of claim 13, wherein the disease is urolithiasis.

16. The method of claim 13, wherein the disease is calcium urolithiasis or struvite urolithiasis.

17. The method of claim 13, wherein the disease is atherosclerosis.

18. The method of claim 13, wherein the disease is microbial plaque or calculus.

19. A method of inhibiting plaque formation on a surface comprising contacting the composition of claim 1 with said surface.

20. The method of claim 19, wherein the composition inhibits growth or formation of a biofilm.

21. The method of claim 19, wherein the surface to be treated is part of medical device.

22. The method of claim 19, wherein the medical device is a prosthetic, stent, or catheter.
